(19) United States
(12) Patent Application Publication
(10) Pub. No.: US 2009/0232900 A1
(43) Pub. Date: Sep. 17, 2009

(54) NANO-MICELLAR PREPARATION OF ANTHRACYLCLINE ANTITUMOR ANTIBIOTICS ENCAPSULATED BY THE PHOSPHATIDE DERIVATIZED WITH POLYETHYLENE GLYCOL

(75) Inventors: Wei Liang, Beijing (CN); Ning Tang, Beijing (CN); Chunling Zhang, Beijing (CN); Zihe Rao, Beijing (CN)

Correspondence Address: KNOBBE MARTENS OLSON & BEAR LLP, 2040 MAIN STREET, FOURTEENTH FLOOR, IRVINE, CA, 92614, US

(73) Assignee: Institute of Biophysics, Chinese Academy of Sciences, Beijing (CN)

(21) Appl. No.: 11/909,885

(22) PCT Filed: Jun. 24, 2005
(86) PCT No.: PCT/CN2005/000919

§ 371 Date: Jul. 24, 2008

(30) Foreign Application Priority Data
Mar. 29, 2005 (CN) 200510059621.8

(51) Int. Cl. A61K 9/51; A61K 31/704; A61K 31/351

(52) U.S. Cl. 424/501; 514/34; 514/459

(57) ABSTRACT

The present invention provides a nano-micellar preparation of anthracycline antitumor antibiotics for intravenous injection, which comprises a therapeutically effective amount of anthracycline antitumor antibiotics, a phosphatide derivatized with polyethylene glycol, together with pharmaceutically acceptable adjuvants. The preparation is prepared by encapsulating the medicament with a nano-micelle to obtain the nano-micellar preparation of anthracycline antitumor antibiotics for injection. The anthracycline antitumor antibiotics and the phosphatide derivatized with polyethylene glycol form a nano-micelle with a highly homogeneous particle size. In the micelle, the hydrophobic core of encapsulated medicament is surrounded by polyethylene glycol molecules to form a hydrophilic protective layer, so that the medicament is prevented from contacting with the enzymes and other protein molecules in blood and being recognized and phagocytozed by reticuloendothelial system in the body, and the circulation time in vivo of the micelle is prolonged.

### FIELD OF THE INVENTION

The present invention relates to a nano-micellar preparation of anthracycline antitumor antibiotics for intravenous injection and method for producing thereof.

### BACKGROUND OF THE INVENTION

Anthracycline antitumor antibiotics is a class of effective broad-spectrum antitumor agent, which is important and widely used in the clinic treatment of various cancers, such as leukemia, lymphoma, breast cancer, lung cancer, liver cancer and many other solid tumors. This class of antitumor agents mainly includes adriamycin (Doxorubicin, ADM), daunorubicin (DNR), epi-adriamycin (Epirubicin, EPI), pirarubicin (THP-ADM), aclacinomycin (ACM). Similar to other cytotoxic antineoplastics, these antitumor agents, however, lack selectivity for tumor tissues and lead to a severe dose-dependent acute toxicity, which is represented clinically as nausea, emesis, alopecia, and inhibition of bone marrow. More severely, the accumulation of drugs in cardiac tissue upon repeated administration will lead to a severe irreversible damage to heart. The toxic side effect of anthracycline antitumor antibiotics greatly limits their clinic application in the long-term treatment for tumors.

One approach to significantly decrease toxicity of anthracycline antitumor antibiotics is to alter their tissue distribution and improve their selectivity for tumor tissues. The liposome preparation of anthracycline antitumor antibiotics could reduce the accumulation of the medicaments in the heart and increase their distribution in tumor tissues, so as to mitigate their dose-dependent acute toxicity. The liposome preparation has been approved for the clinic treatment of various types of cancer and a satisfying therapeutical effect has been achieved. The liposome preparations of anthracycline antitumor antibiotics available on market include adriamycin liposome and daunorubicin liposome. In addition, two liposomal products, amphotericin liposome and paclitaxel liposome, have been approved by State Drug Administration. The liposome preparations of anthracycline antitumor antibiotics, however, also suffer from many disadvantages. For example, the medicament is encapsulated in inner water phase and could play its role only after being released from the liposome. The minimal size of the liposome is 50 nm and the entry of the liposome into cells is completed via fusion and pinocytosis mechanism. Thus, the cytotoxic effect of the medicament encapsulated in liposome is weaker than that of free medicament. The production process of the liposome is complicated and the complexing of several lipid components (at least two lipid components) is required, wherein special equipments and devices are required to control the particle size. In addition, flocculation occurs frequently during the storage.

In water, amphiphatic molecules will congregate spontaneously to form micelle when the concentration of the molecules exceeds critical micelle concentration. Taking advantage of this property, medicament is encapsulated in the hydrophobic core of the micelle. Micellar preparations have been used in clinic treatment practice for a long time. For example, deoxysodium cholate is utilized to solubilize amphotericin B and the like. A paper titled with “Polymer micelle: a novel drug carrier” by Kun et al. summarizes the use of micelle as a drug carrier (Adv. Drug. Del. Rev. 21:107-116, 1976). Recently, as a targeting, long-circulating, sustained release drug carrier, polymer micelle has drawn great attention of people and becomes the hotspot of drug delivery system. Yokoyama et al. employs a polymer to encapsulate antitumor drug and investigates its activity against solid tumor and cytotoxicity as well as its long-circulating property in blood, wherein the polymer is capable of forming micelle (Cancer Res. 51:3229-3236, 1991). Lipids modified with PEG-phospholipid have been demonstrated to be characterized by their long circulation in animal and human body, and can be safely used in clinic treatment (Gregoriadis, G. 1995 TIBTECH, 13:527-53). As a carrier for drugs with poor solubility, polyethylene glycol-phospholipid micelle has been comprehensively summarized by investigators (Torchilin, V. P. J. Controlled Release, 73:137-172).

Polyethylene glycol (PEG) is a water-soluble polymer stable under physiological condition. Because the space structure of PEG is capable of preventing the approach of plasma proteins, PEG has been widely used to modify the properties of phospholipid and protein drugs. In nanoparticle delivery system, PEG is capable of forming a hydrophilic protection layer on the surface of particles to prevent the aggregation of the particles, avoiding being recognized and phagocytized by reticuloendothelial system in body, and extending the retention time of drugs in blood circulation, whereby a long circulation is achieved.

Nano-micelle prepared from a phospholipid derivatized with polyethylene glycol possesses advantages over general nanoparticles. The particle size of the nano-micelle is small and substantially between 10 nm and 50 nm. The nano-micelle is a dynamically stable system, which on one hand avoids the disadvantage of other microparticle delivery system, i.e. easy to aggregate, and on the other hand reaches lesion sites more easily, whereby the drug distribution is improved and the targeting of drug for tumor tissue is increased.

### SUMMARY OF THE INVENTION

One objective of the present invention is to provide a nano-micellar preparation of anthracycline antitumor antibiotics for intravenous injection, which is a dynamically stable system, has good stability and can be used in targeted therapy in vivo. Thus, the nano-micellar preparation is capable of improving the drug distribution in tumor tissues, increasing effectiveness and decreasing toxicity.

Another objective of the present invention is to provide a method of producing the nano-micellar preparation of anthracycline antitumor antibiotics for intravenous injection.

The present invention provides a nano-micellar preparation of anthracycline antitumor antibiotics for intravenous injection, comprising a therapeutically effective amount of anthracycline antitumor antibiotics, a phosphatide derivatized with polyethylene glycol, together with pharmaceutically acceptable adjuvants.

In one embodiment, a nano-micellar preparation of anthracycline antitumor antibiotics is provided, which is produced by a suitable preparation method from basic adjuvant, a phosphatide derivatized with PEG.

### DETAILED DESCRIPTION OF INVENTION

The present invention provides a nano-micellar preparation of anthracycline antitumor antibiotics for intravenous injection, which comprises anthracycline antitumor antibiotics, a phosphatide derivatized with polyethylene glycol, together with pharmaceutically acceptable adjuvants.

According to the present invention, the molar ratio of the anthracycline antitumor antibiotics and the phosphatide derivatized with polyethylene glycol is between 1:0.5 and 1:10, preferably between 1:1 and 1:3.

In the present invention, the anthracycline antitumor antibiotics is one or more medicaments selected from the group consisted of adriamycin, daunorubicin, epi-adriamycin, pirarubicin and aclacinomycin.

In one embodiment, the phosphatide derivatized with polyethylene glycol is formed by coupling polyethylene glycol molecule to the nitrogenous bases on the phospholipid molecule through a covalent bond.

In another embodiment, the phosphatide according to present invention is a phosphatide derivatized with polyethylene glycol, wherein the fatty acid in the phosphatide portion comprises 10 to 24 carbon atoms, preferably 12, 14, 16, 18, 20, 22 and 24 carbon atoms. The fatty acid chain may be saturated or partially saturated. In particular, the fatty acid may be lauric acid (C12), myristic acid (C14), palmitic acid (C16), stearic acid or oleic acid or linoleic acid (C18), arachidic acid (C20), behenic acid (C22) or lignocerate (C24).

In still another embodiment, the phosphatide portion may be phosphatidylethanolamine (PE), phosphatidylcholine (PC), phosphatidylinositol (PI), phosphatidylserine (PS), diphosphatidyl glycerol, acetal phosphatide, lysophosphatidylcholine (LPC), or lysophosphatidyl ethanolamine (LPE).

In another aspect, the phosphatide in the phosphatide derivatized with polyethylene glycol is preferably phosphatidylethanolamine, and more particular, distearyl phosphatidylethanolamine, dipalmitoyl phosphatidylethanolamine, dioleoyl phosphatidylethanolamine.

The polyethylene glycol in the phosphatide derivatized with polyethylene glycol has a molecular weight of between 200 and 20000 daltons (depending on the number of ethoxy group in the long chain of PEG), preferably between 500 and 10000, more preferably between 1000 and 10000 (the number of ethoxy group is 22 to 220), and most preferably 2000.

In a preferred embodiment, the phosphatide derivatized with polyethylene glycol according to present invention is distearyl phosphatidylethanolamine derivatized with polyethylene glycol 2000.

The nano-micellar preparation of anthracycline antitumor antibiotics according to present invention, as required, may be a solution or in a lyophilized form.

In the nano-micellar preparation of anthracycline antitumor antibiotics according to present invention, the micelle has a size range of 5-100 nm, preferably 10-50 nm, most preferably 10-20 nm. The use level of the anthracycline antitumor antibiotics is 1-10 mg per ml preparation, preferably 1-3 mg per ml preparation, and the use level of the phosphatide derivatized with polyethylene glycol is 1-500 mg per ml preparation, preferably 10-30 mg per ml preparation.

In still another aspect, the phosphatide derivatized with polyethylene glycol is formed by coupling polyethylene glycol molecule to the phospholipid molecule through a covalent bond.

The nano-micellar preparation of anthracycline antitumor antibiotics according to present invention utilizes a phosphatide derivatized with polyethylene glycol alone or in combination with other phosphatides as carrier, wherein a therapeutically effective amount of anthracycline antitumor antibiotics is encapsulated in the formed nanomicelle by a particular preparation process. When necessary, a antioxidant, osmotic pressure adjusting agent, or pH adjusting agent may be added.

In still another aspect, the micellar preparation comprises anthracycline antitumor antibiotics, an amphiphatic molecule and a pharmaceutically acceptable antioxidant, osmotic pressure adjusting agent, or pH adjusting agent. The amphiphatic molecule may be a phosphatide derivatized with polyethylene glycol or other phosphatides. Other phosphatides include phosphatidic acid, phosphatidylinositol, phosphatidylserine, phosphatidyl glycerol, cardiolipin, soyabean lecithin, phosphatidylcholine, phosphatidylethanolamine, hydrolecithin etc.

In the micellar preparation according to present invention, the molar percentage of the phosphatide derivatized with PEG in total phosphatide is in the range of 20% to 100%, preferably 60% to 100%.

The final micellar preparation may be a solution, which comprises 1 mg/ml to 10 mg/ml of anthracycline antitumor antibiotics, 1 mg/ml to 500 mg/ml of total phosphatide. The concentration of other additives is 0.01% to 5%.

The final micellar preparation may be a lyophilized powder, which comprises 0.02% to 50% by weight of anthracycline antitumor antibiotics, 50% to 95% by weight of total phosphatide and 10% to 90% by weight of other additives.

Because both anthracycline antitumor antibiotics and phosphatides are easily oxidized, the micellar preparation of anthracycline antitumor antibiotics according to present invention may further comprise an antioxidant, such as water soluble antioxidant (ascorbic acid, sodium bisulphate, EDTA, use level: 0.01 to 1.0 wt %) and fat soluble antioxidant (tocopherol, BHA, propyl gallate, use level: 0.01 to 1.0 wt %).

As required, pH adjusting agent (various buffer system, such as citric acid-sodium citrate, acetic acid-sodium acetate, phosphate etc.) may be added to the micellar preparation according to present invention with a use level of 1 mM to 100 mM. The medicament solution is adjusted to a pH of 3.0 to 8.0, more preferably 6 to 7.5.

As required, an osmotic pressure adjusting agent (sodium chloride, glucose, mannitol) may be added to the micellar preparation according to present invention. The osmotic pressure adjusting agent may be various pharmaceutically acceptable salts and carbohydrates for adjusting osmotic pressure to be isotonic to or somewhat higher than that of human body (the osmotic pressure range of human body is 290-310 mmol/L).

The invention further provides a method of producing the nano-micellar preparation of anthracycline antitumor antibiotics, comprising: encapsulating the anthracycline antitumor antibiotics in a nanomicelle formed with a phosphatide derivatized with polyethylene glycol so as to prepare the nano-micellar preparation of anthracycline antitumor antibiotics for intravenous injection.

In one particular embodiment, the method of producing the nano-micellar preparation of anthracycline antitumor antibiotics according to present invention includes the following steps:
(1) dissolving the anthracycline antitumor antibiotics and the phosphatide derivatized with polyethylene glycol in an organic solvent;
(2) removing the organic solvent so as to obtain a polymer lipid film containing the anthracycline antitumor antibiotics;
(3) adding water or a buffer solution to the polymer lipid film obtained in step (2), and hydrating at a temperature between 25° C. and 60° C.;
(4) vortexing by shaking or ultrasonic processing to obtain the nanomicelle of phosphatide derivatized with polyethylene glycol, the anthracycline antitumor antibiotics being encapsulated therein.

The organic solvent in step (1) of the method according to present invention is methanol, ethanol, chloroform, or the mixtures thereof.

The organic solvent is removed under reduced pressure and/or under vacuum condition in step (2) of the method according to present invention.

The buffer solution in step (3) of the method according to present invention is citrate or phosphate buffer solution.

The hydrating in step (3) of the method according to present invention is performed in water bath at a temperature between 25° C. and 60° C., preferably between 35° C. and 45° C., for 1 to 2 hours.

The vortexing by shaking or ultrasonic processing in step (4) of the method according to present invention is conducted for 1 to 5 minutes.

In one embodiment, the method according to present invention further comprises adjusting the pH of the obtained micelle solution to 3.0-8.0, preferably 6.5-7.4, with a pH adjusting agent.

In another embodiment, the method according to present invention further comprises lyophilizing the obtained micelle solution to produce a lyophilized preparation.

In details, the micellar preparation according to present invention is produced by the following procedures: dissolving the anthracycline antitumor antibiotics and the phosphatide derivatized with polyethylene glycol in an organic solvent in a leptoclados-type bottle; volatilizing the organic solvent to dryness with a rotary evaporator so as to form a thin uniform lipid film on the surface of the leptoclados-type bottle; dissolving a water soluble additive (water soluble antioxidant, osmotic pressure adjusting agent, pH adjusting agent) in water and the water solution is added to the leptoclados-type bottle and hydration is performed by shaking; filtering through 0.22 μm microfiltration membrane for filtration sterilization to produce the micellar preparation of anthracycline antitumor antibiotics for intravenous injection. The particle size of the formed nanomicelle is in the range of 10-50 nm, preferably 10-30 nm. As required, the preparation may be a suspension or in a lyophilized form.

For the purpose of better understanding of the invention, several technical terms are defined as follows.

“Micelle” refers to an amphiphatic molecule which is capable of congregating spontaneously to form micelle when the concentration of the molecules in water solution exceeds critical micelle concentration. The structure of the micelle differs from that of liposome in that the micelle does not possess a lipid bilayer structure. In general, in the structure of micelle, hydrophobic portion orients toward inner to form a hydrophobic core, while hydrophilic portion orients toward outside to form a hydrophobic surface. The particle size of micelle is small and on average about 10-20 nm. Therefore, micelle is not only a thermodynamically stable system, but also a dynamically stable system. In addition, the micelle particle does not congregate and stratify easily and its loading capability is high, even when the drug concentration is low.

“Phosphatide”, the molecular structure of phosphatide is similar to that of fat and differs in that only two fatty acids is linked to the glycerol molecule in phosphatide and the third hydroxyl group is coupled with phosphoric acid to form ester. With such a structure, phosphatide enables itself an amphiphatic molecule, wherein its phosphoric acid or phosphoric acid ester terminus is polar and easy to attract water to constitute a hydrophilic head of the phosphatide molecule, while its fatty acid terminus is nonpolar, not attracted by water, and form a hydrophobic tail of the phosphatide molecule. The main phosphatide involved in the invention is phosphatide derivatized with polyethylene glycol. In instant invention, the phosphatide derivatized with polyethylene glycol may also be used in combination with other phosphatides.

“Therapeutically effective amount” refers to the amount of the anthracycline antitumor antibiotics when a therapeutic effect is produced. According to the invention, the unit dosage of anthracycline antitumor antibiotics is 5-100 mg, preferably 10-20 mg, most preferably 20 mg, and can be modified according to individual requirement of each subject.

The nano-micellar preparation of anthracycline antitumor antibiotics according to present invention utilizes polyethylene glycol (PEG) as main base and is capable of preventing the nano-micellar preparation from being phagocytized by reticuloendothelial system in body. Thus, the retention time of the nanomicelle in blood circulation is prolonged and the dynamical property of the drug in body (drug distribution) is improved, so that the effectiveness is increased and toxicity is decreased.

As described above, anthracycline antitumor antibiotics lead to a severe dose-dependent acute toxicity and lack selectivity for tumor tissues. Conventional injection solution of anthracycline antitumor antibiotics, upon being injected into body, results in an accumulation of the drugs in cardiac tissue, which in turn leads to a severe irreversible damage to heart. The toxic side effect of anthracycline antitumor antibiotics greatly limits their clinic application in the long-term treatment for tumors. Liposomes of anthracycline antitumor antibiotics could reduce the accumulation of the drugs in the heart, increase the drug distribution in tumor tissues, reduce the dose-dependent acute toxicity and thus has been approved for the clinic treatment of various types of cancer and a satisfying therapeutical effect has been achieved. Liposomes of anthracycline antitumor antibiotics, however, also suffer from many disadvantages. For example, the medicament is encapsulated in inner water phase and could play its role only after being released from the liposome. The minimal size of the liposome is 50 nm and the entry of the liposome into cells is completed via fusion and pinocytosis mechanism. Thus, the cytotoxic effect of the medicament encapsulated in liposome is weaker than that of free medicament. The production process of the liposome is complicated and the complexing of several lipid components (at least two lipid components) is required, wherein special equipments and devices are required to control the particle size. In addition, flocculation occurs frequently during the storage.

To overcome the disadvantages of above preparations, the present invention utilizes a phosphatide derivatized with polyethylene glycol alone or in combination with other phosphatides as main carrier to produce the micelle preparation of anthracycline antitumor antibiotics, wherein the encapsulation percentage exceeds 90%. The major technological advantage of present invention is the utilization of phosphatide derivatized with polyethylene glycol, which is capable of spontaneously forming a nanomicelle with a very uniform particle size. The size of the nanomicelle is in a range of 10-30 nm.

In the micelle, the hydrophobic core of encapsulated medicament is surrounded by polyethylene glycol molecules to form a hydrophilic protective layer, so that the medicament is prevented from contacting with the enzymes and other protein molecules in blood and being recognized and phagocytozed by reticuloendothelial system in body, and the circulation time in vivo of the micelle is prolonged. The encapsulation of the medicament in the hydrophobic core of micelle prevents the medicament from being destroyed by external factors (water, oxygen, light) and improves significantly the stability of the medicament during storage. Furthermore, the micelle is capable of altering the dynamical property of drug (drug distribution) in vivo, increasing the drug distribution in tumor tissues and thereby improving efficacy and decreasing toxicity.

The following examples are intended to illustrate the invention, but are in no way intended to limit the scope thereof.

### BRIEF DESCRIPTION OF THE DRAWINGS

FIG. 1 illustrates a cytotoxicity assay in vitro of adriamycin micellar preparation;

FIG. 2 illustrates a tumor growth inhibition assay in vivo of adriamycin micellar preparation.

### MODE FOR CARRYING OUT THE INVENTION

### Example 1

### Production of Nano-Micellar Preparation of Anthracycline Antitumor Antibiotics

The formulation of the preparation is listed in Table 1:

TABLE 1
The formulation of the nano-micellar preparation
of anthracycline antitumor antibiotics

| | Lipids/ | Medi- | |
|---|---|---|---|
| Medi- | Medicament | cament | |
| cament | (mol/mol) | (mg/ml) | Hydrated solution |
| ADM | 2:1 | 2 | Phosphate buffer solution, pH 7.0 |
| DNR | 2:1 | 2 | Phosphate buffer solution, pH 7.0 |
| EPI | 2:1 | 2 | Phosphate buffer solution, pH 7.0 |
| THP-ADM | 2:1 | 2 | Phosphate buffer solution, pH 7.0 |
| ACM | 2:1 | 2 | Phosphate buffer solution, pH 7.0 |

Preparation process: ADM, DNR, EPI, THP-ADM and ACM with a ratio according to above formulation were dissolved in ethanol (1-5 mg/ml). In addition, PEG 2000 distearyl phosphatidylethanolamine (PEG2000-DSPE) was weighed, dissolved in a suitable amount of chloroform, and then placed into a 100 ml leptoclados-type bottle. The organic solvent was volatilized with a rotary evaporator so as to form a thin uniform phosphatide film on the surface of the leptoclados-type bottle. A phosphate buffer solution was added to the leptoclados-type bottle and hydration was performed by shaking at 37° C. under the protection of nitrogen atmosphere for 1 hour. 0.22 μm microfiltration membrane was used for filtration sterilization to produce the micellar preparation of anthracycline antitumor antibiotics for intravenous injection. The obtained sample was a clear suspension with a tangerine appearance, and had an average particle size of 15 nm with a size distribution between 10 nm and 20 nm. The encapsulation percentage was over 90%.

### Example 2

### The Encapsulation Percentage of ADM-PEG2000-DSPE Micelle

The formulation of the preparation is listed in Table 2:

TABLE 2
The encapsulation percentage of ADM-PEG2000-DSPE micelle

| | | | Encap- |
|---|---|---|---|
| Lipids/ | Medi- | | sulation |
| Medicament | cament | | percentage |
| (mol/mol) | (mg/ml) | Hydrated solution | (%) |
| 05:1 | 2 | Phosphate buffer solution, pH 7.0 | 70 |
| 1:1 | 2 | Phosphate buffer solution, pH 7.0 | 92 |
| 2:1 | 2 | Phosphate buffer solution, pH 7.0 | 97 |
| 5:1 | 2 | Phosphate buffer solution, pH 7.0 | 99 |
| 10:1 | 2 | Phosphate buffer solution, pH 7.0 | 99 |

Preparation process: According to Lipids/Medicament ratios in above formulation, ADM was weighed and dissolved in ethanol (2 mg/ml). PEG2000-DSPE was weighed and dissolved in a suitable amount of chloroform, and then placed into a 100 ml leptoclados-type bottle. The organic solvent was volatilized with a rotary evaporator so as to form a thin uniform phosphatide film on the surface of the leptoclados-type bottle. A phosphate buffer solution was added to the leptoclados-type bottle and hydration was performed by shaking at 37° C. under the protection of nitrogen atmosphere for 1 hour. 0.22 μm microfiltration membrane was used for filtration sterilization to produce the micellar preparation of adriamycin for intravenous injection. The obtained sample was a clear solution with a tangerine appearance, and had an average particle size of 15 nm with a size distribution between 10 nm and 20 nm.

### Example 3

### The Production of Daunorubicin Micellar Preparation

The formulation of the preparation is listed in Table 3:

TABLE 3
The formulation of adriamycin micellar preparation

| Components | Concentration (mM) |
|---|---|
| DNR | 3.68 |
| PEG2000-DPPE | 4.9 |
| PG | 2.46 |
| VE | 0.1 |
| EDTA | 0.02 |
| Water | 100 ml |

Preparation process: According to above formulation, DNR was weighed and dissolved in ethanol (2 mg/ml). PEG2000-DPPE, phosphatidyl glycerol (PG) and tocopherol (VE) were weighed and dissolved in a suitable amount of chloroform, and then placed into a 100 ml leptoclados-type bottle. The organic solvent was volatilized with a rotary evaporator so as to form a thin uniform phosphatide film on the surface of the leptoclados-type bottle. An EDTA aqueous solution was added to the leptoclados-type bottle and hydration was performed by shaking at 37° C. under the protection of nitrogen atmosphere for 1 hour. 0.22 μm microfiltration membrane was used for filtration sterilization to produce the micellar preparation of daunorubicin for intravenous injection. The obtained sample was a clear suspension with a tangerine appearance, and had an average particle size of 15 nm with a size distribution between 10.0 nm and 20 nm. The encapsulation percentage was over 90%. The above micelle solution could be lyophilized to obtain a lyophilized powder.

### Example 4

### Cytotoxicity Assay In Vitro of Adriamycin Micellar Preparation

A cytotoxicity assay in vitro and a tumor growth inhibition assay in vivo were used to verify the antitumor effect of the nano-micellar preparation of anthracycline antitumor antibiotics.

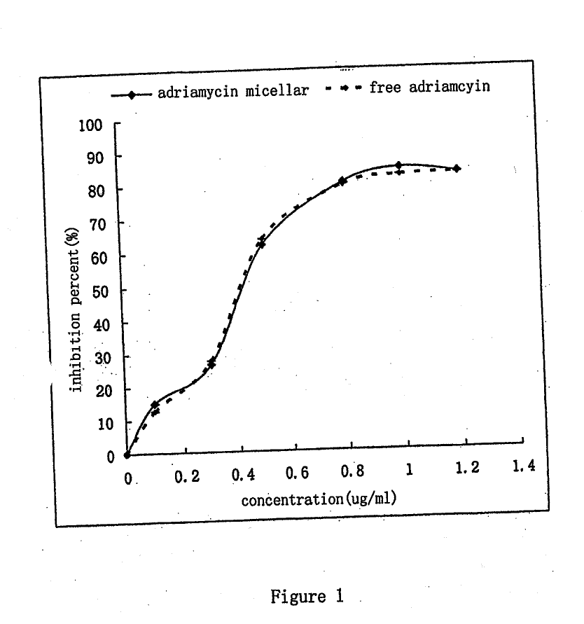

A549 cells were inoculated on a 96-well plate (8.0×10³/well) and incubated overnight. Culture media was then washed out and 5 μl samples with various concentrations of adriamycin (both free adriamycin and adriamycin encapsulated in PEG-distearyl phosphatidylethanolamine micelle) were added in triplicate respectively. To each well was added 100 μl medium supplemented with 10% fetal calf serum, and the cells were grown in an incubator (37° C., 5% CO₂) for further 24 hours or 48 hours. Cells were taken out at each setting time points and added with 20 μl MTT (5 mg/ml). After incubation for further 4 hours, each well was added with 150 μl DMSO for dissolution and then placed into a Micro-Plate Reader to read out its maximum absorption at 590 nm. The growth curve was plotted for each concentration and shown in FIG. 1.

### Example 5

### Tumor Growth Inhibition Assay In Vivo of Adriamycin Micellar Preparation

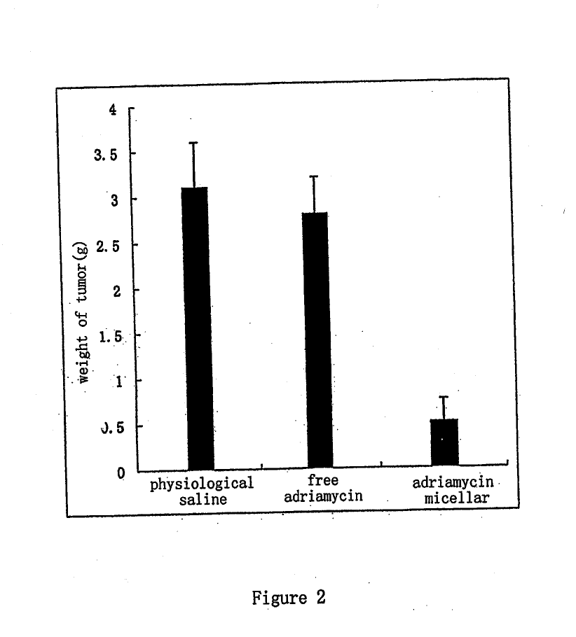

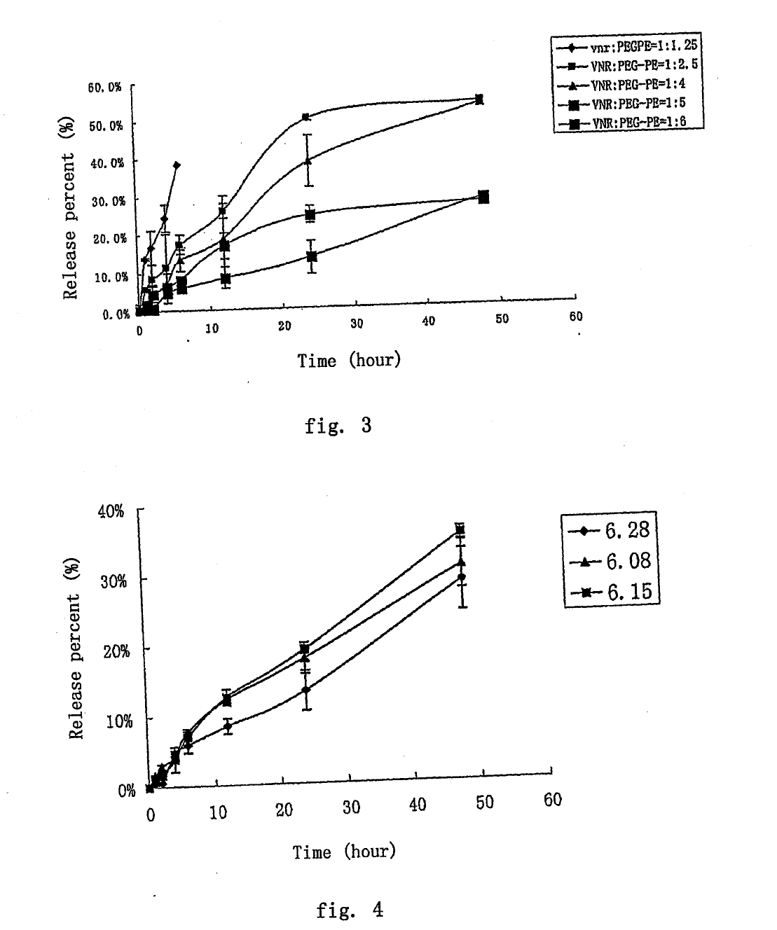

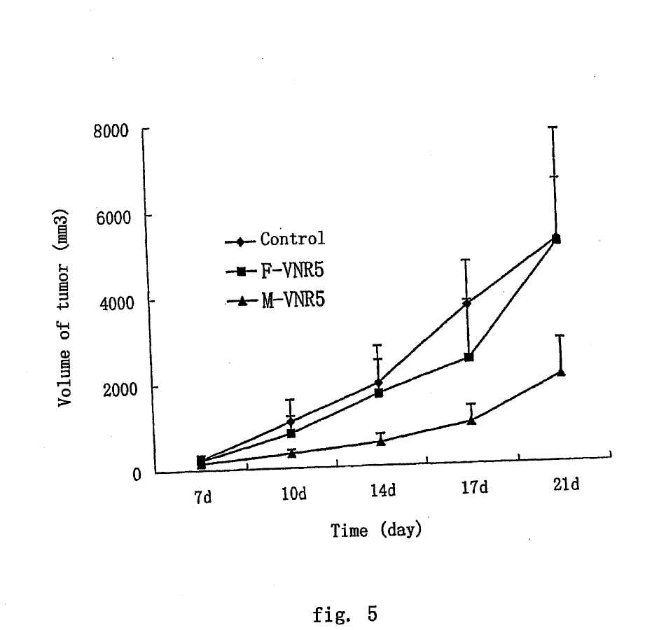

Living mice bearing Lewis lung tumor were sacrificed through dislocation. Skin was sterilized with iodine tincture and 75% ethanol was used for deiodination. Tumor was peeled off, placed in sterile physiological saline, and ground. Each mouse was subcutaneously inoculated on back with 0.2 ml tumor cell. The mice loaded with tumor were then divided into three groups, 10 mice each group. Group I was the HCl adriamycin solution (5 mg/ml) group (5.0 mg/kg); Group II was the adriamycin nanomicelle (5 mg/ml) group, wherein the molar ratio of adriamycin to PEG2000 distearyl phosphatidylethanolamine was 1:2, 5.0 mg/kg; and Group III was physiological saline control group, 0.2 ml/mouse. On the third day after the tumor inoculation, administration was conducted once via caudal vein. The volume of tumor and the weight of mice were measured daily after the administration. On the fifteen day after the administration, the mice were sacrificed and tumors were peeled off and weighed. The results were shown in FIG. 2.

1. A nano-micellar preparation of anthracycline antitumor antibiotics for intravenous injection, which comprises anthracycline antitumor antibiotics, a phosphatide derivatized with polyethylene glycol, together with pharmaceutically acceptable adjuvants.

2. The micellar preparation of claim 1, wherein the molar ratio of the anthracycline antitumor antibiotics and the phosphatide derivatized with polyethylene glycol is between 1:0.5 and 1:10.

3. The micellar preparation of claim 1, wherein the anthracycline antitumor antibiotics is one or more medicaments selected from the group consisting of adriamycin, daunorubicin, epi-adriamycin, pirarubicin and aclacinomycin.

4. The micellar preparation of claim 1, wherein the phosphatide derivatized with polyethylene glycol is formed by coupling polyethylene glycol molecule to the nitrogenous bases on the phospholipid molecule through a covalent bond.

5. The micellar preparation of claim 4, wherein the fatty acid in the phosphatide part of the phosphatide derivatized with polyethylene glycol comprises 10 to 24 carbon atoms, and the fatty acid chain is saturated or partially saturated.

6. The micellar preparation of claim 4, wherein the phosphatide in the phosphatide derivatized with polyethylene glycol is phosphatidylethanolamine, phosphatidylcholine, phosphatidylinositol, phosphatidylserine, diphosphatidyl glycerol, acetal phosphatide, lysophosphatidylcholine, or lysophosphatidyl ethanolamine.

7. The micellar preparation of claim 6, wherein the phosphatide in the phosphatide derivatized with polyethylene glycol is distearyl phosphatidylethanolamine, dipalmitoyl phosphatidylethanolamine, or dioleoyl phosphatidylethanolamine.

8. The micellar preparation of claim 4, wherein the polyethylene glycol in the phosphatide derivatized with polyethylene glycol has a molecular weight of between 200 and 20000 daltons.

9. The micellar preparation of claim 4, wherein the phosphatide derivatized with polyethylene glycol is distearyl phosphatidylethanolamine derivatized with polyethylene glycol 2000.

10. The micellar preparation of claim 1, wherein the micellar preparation is a suspension or in a lyophilized form.

11. The micellar preparation of claim 1, wherein the pharmaceutically acceptable adjuvant is a pharmaceutically acceptable antioxidant, osmotic pressure adjusting agent, or pH adjusting agent.

12. The micellar preparation of claim 11, wherein the pH adjusting agent is citric acid-sodium citrate, acetic acid-sodium acetate, or phosphate, or the combination thereof.

13. A method of producing the nano-micellar preparation of anthracycline antitumor antibiotics for intravenous injection according to claim 1, comprising: encapsulating the anthracycline antitumor antibiotics in a nanomicelle formed with a phosphatide derivatized with polyethylene glycol so as to prepare the nano-micellar preparation of anthracycline antitumor antibiotics for intravenous injection.

14. The method of claim 13, comprising the following steps:
(1) dissolving the anthracycline antitumor antibiotics and the phosphatide derivatized with polyethylene glycol in an organic solvent;
(2) removing the organic solvent so as to obtain a polymer lipid film containing the anthracycline antitumor antibiotics;
(3) adding water or a buffer solution to the polymer lipid film obtained in step
(2), and hydrating at a temperature between 25° C. and 60° C.;
(4) vortexing by shaking or ultrasonic processing to obtain the nanomicelle of phosphatide derivatized with polyethylene glycol, the anthracycline antitumor antibiotics being encapsulated therein.

15. The method of claim 14, wherein the organic solvent in step (1) is methanol, ethanol, chloroform, or the mixtures thereof.

16. The method of claim 14, wherein the organic solvent is removed under reduced pressure or under vacuum condition in step (2).

17. The method of claim 14, wherein the buffer solution in step (3) is citrate or phosphate buffer solution.

18. The method of claim 14, wherein the hydrating in step (3) is performed in water bath at a temperature between 25° C. and 60° C., preferably between 35° C. and 45° C., for 1 to 2 hours.

19. The method of claim 14, wherein the vortexing by shaking or ultrasonic processing in step (4) is conducted for 1 to 5 minutes.

20. The method of claim 14, further comprising: adjusting the pH of the obtained micelle solution to 3.0-8.0, with a pH adjusting agent.

21. The method of claim 13, further comprising: lyophilizing the obtained micelle suspension to produce a lyophilized preparation

22. The micellar preparation of claim 5, wherein the fatty acid is selected from the group consisting of lauric acid, myristic acid, palmitic acid, stearic acid, oleic acid, linoleic acid, arachidic acid, behenic acid and lignoceric acid.

23. The micellar preparation of claim 8, wherein the polyethylene glycol in the phosphatide derivatized with polyethylene glycol has a molecular weight of between 500 and 10000 daltons.

24. The micellar preparation of claim 8, wherein the polyethylene glycol in the phosphatide derivatized with polyethylene glycol has a molecular weight of between 1000 and 10000 daltons.

25. The micellar preparation of claim 8, wherein the polyethylene glycol in the phosphatide derivatized with polyethylene glycol has a molecular weight of 2000 daltons.

26. The method of claim 18, wherein the hydrating in step (3) is performed in water bath at a temperature between 35° C. and 45° C., for 1 to 2 hours.

27. The method of claim 14, further comprising: adjusting the pH of the obtained micelle solution to 6.5-7.4, with a pH adjusting agent.
